(19) United States
(12) Patent Application Publication
(10) Pub. No.: US 2005/0065537 A1
(43) Pub. Date: Mar. 24, 2005

(54) Surgicals metals with improved hardness and methods for making same

(76) Inventors: Vincent C Tangherlini, Rancho Santa Marganita,, CA (US); Russell E Ahlberg, Rancho Santa Margarita, CA (US); Arkadiusza A Strokosz, Dana Point, CA (US); Gary M Johnson, Mission Viejo, CA (US); Matthew A Wixey, Dana Point, CA (US); Linda C Phinuwong, Garden Grove, CA (US)

Correspondence Address: Kenneth K Vu, 22872 Avenida Empressa, Rancho Santa Margarita, CA, 92688, US

(21) Appl. No.: 10/479,351

(22) PCT Filed: May 30, 2002
(86) PCT No.: PCT/US02/17332

(30) Foreign Application Priority Data
Jun. 5, 2001 (US) 60296264

(51) Int. Cl. A61B 17/08

(52) U.S. Cl. 606/157

(57) ABSTRACT

A surgical device includes a structural portion which is formed of a first element, such as titanium, which has an outer surface and an inner core. A second element, such as oxygen, is combined with the first element without creating a shear plane, to create a concentration of the second element in the first element which varies from the outer surface to the inner core. In an associated method, the second element is driven into the first element under other than ambient conditions to develop a concentration gradient of the second element between the outer surface and the inner core of the first element.

### BACKGROUND OF THE INVENTION

1. Field of the Invention

This invention relates generally to surgical devices formed of metals and to methods for improving the hardness of surgical metals.

2. Discussion of Relevant Art

Safety and efficacy are of primary concern with respect to the use of all surgical instruments and devices. Where surgical metals are included in these instruments and devices, increased hardness for a given size may offer significant advantages. However, if ductility is desired, the increased hardness must be balanced against the need for bendability.

Consider, for example, surgical clips and staples. Clips are typically used to occlude or ligate body conduits such as blood vessels. Staples usually function to close wounds or otherwise bring tissue together to promote healing. As used herein, the word “clip” shall refer to both clips and staples, as well as any other medical device formed of wire, such as a spring.

With respect to efficacy, a clip needs to have a size and shape that permits easy access to the operative site. It must be bendable at the operative site to engage tissue and must otherwise perform its intended function at the operative site. Finally, the clip must remain at the operative site and continue performing that function until it is removed or its function is no longer desired.

These criteria speak mainly to the ability of the clip to accomplish its function. For example, if the clip is intended to occlude a blood vessel, it must be small enough to facilitate insertion into the body to gain access to the blood vessel. At the operative site, the clip must be capable of engaging the blood vessel where it can then be bent to pinch the blood vessel and thereby occlude blood flow. Continued effectiveness relies on the ability of the clip to remain at the operative site and to occlude blood flow until the clip is removed.

With respect to safety, some of these same factors come into play. For example, it is important that the clip remain where it is placed so that it does not fall into a body cavity. This can be of particular concern in open procedures where there is generally more movement of hands and organs around the operative site, and consequently a greater probability of dislodging the clip.

The clips of the past have been provided with a size facilitating insertion and placement. The clips have also been provided in different forms, but in general they have a U-shape configuration with a back section connecting opposing leg sections that initially extend in a common direction. In some configurations, a locking element is provided at the free end of each leg portion, but this feature tends to complicate ultimate removal of the clip

By comparison, clips configured with leg sections having free ends are more easily removed, but rely to a greater extent on the strength of the surgical metal for occlusion and retention. Thus, for a given cross section, a metal wire with increased strength will grip the tissue, such as the blood vessel, with a greater force. This will facilitate retention of the clip, meaning that it will have increased properties for staying in place, as well as better performance of the intended function, such as occlusion.

In U.S. Pat. No. 4,188,953 Klieman, et al. disclosed a clip which its uncrimped state is typical of many of the clips in that it includes a back section and opposing leg sections which initially extend generally in a common direction.

A clip disclosed by Chen et al. in U.S. Pat. No. 5,160,339, is also worthy of note. This clip includes a resilient hinge means at the proximal ends of the legs and a latch means at the distal ends of the legs. It will be noted that the latch or locking means provides increased retention but tends to inhibit removability of the clips. In the absence of a latch or locking means, clips rely even more heavily on the strength of the surgical metal for retention.

Clips of the past, have been made of biocompatible materials, and have typically been formed of titanium. This metal is non-ferromagnetic and consequently does not interfere with magnetic resonance imaging. Titanium is also more stable than stainless steel. Many of these advantages can be achieved by other metals or metal alloys which might also be used for clips.

Titanium is known to be highly reactive with oxygen. In fact, titanium in air will rapidly form a naturally occurring, micro-thin layer of titanium oxide on its surface. This natural layer functions as a barrier against further oxygen penetration so the titanium tends to remain relatively pure and inert with this micro-thin oxidation layer.

Manufacturers of titanium medical devices have gone to great lengths to maintain the material in its “as bought” condition. Thus, when the metal is purchased as titanium Grade 1, every effort has been made to maintain the ductility and other properties associated with that grade. Bending operations which induce stress into the material have typically been followed by high temperature heating to relieve those stresses. In order to avoid altering the physical properties and chemical makeup of the titanium, this heating has been done in a vacuum or inert gas.

This is only one example of the vast teachings of the past which encourage one to avoid oxygen penetration beyond the natural layer. In these teachings, the coating is commonly referred to as “corrosion” and several methods are offered either to avoid the “corrosion” or eliminate the “corrosion” once it has occurred. In many settings, pride is taken in the ability to heat titanium without developing this corrosive layer. Hence, in the procedures of the past, a vacuum has been strictly and laboriously applied in order to limit exposure to gases during the heating step.

The manufacturing steps are all undertaken with the intent of providing the clip with desirable properties which can optimize its performance in the operative environment. One of these desirable properties is an ability to absorb mending stresses during crimping so that the clip does not tend to open slightly when the placement force is relieved. This phenomena, commonly referred to as spring-back, results when the stresses induced by the crimping force cause the clip to open when the crimping force is removed.

Another property of particular interest relates to the holding force which opposes removal of the clip from a vessel, for example. It is desirable to maintain this holding force relatively high in order that the clip can provide the desired inclusion inoperatively disposed. Unfortunately, this holding force is typically reduced when the clip has a high degree of bendability.

A pull test can be performed to measure the holding strength of a particular clip design. In such a test, a clip is crimped onto a vessel or tube and the back section of the clip engaged by a small wire or other tension element. When this tension element is pulled in a direction parallel to the crimped legs of the clip, the force required to remove the clip can be measured to provide a relative indication of holding.

Another test which would demonstrate the advantages of the present invention is a micro-hardness test. In this test, a diamond, the hardest material known to man, is pressed with a known force against the target material. Measurement of the resulting indentation provides a quantifiable measurement of the hardness of the material.

Thus, this micro-hardness test can be used to measure the hardness at any point on the cross-sectional area of the clip wire. For example, hardness can be measured at the outer surface of the wire as well as at the core of the wire or any point-therebetween.

Suffice it to say, that with respect to clips, greater strength for a given size is always desirable where sufficient ductility remains to facilitate bending the clip into its operative form. This is particularly true for open configurations of clips where retention is even more dependent upon strength.

### SUMMARY OF THE INVENTION

In accordance with the present invention, the strength of surgical metals can be significantly increased. With respect to clips and other wire devices, the increased strength will provide better strength, reduced bendability and higher retention. Compared to clips of the past, the clips of the present invention will hold the vessels tighter, given the same cross-sectional area. With greater strength, one might expect that there would be increased spring-back. This does not appear to be the case as the spring-back is actually reduced with this process. As a result, retention is facilitated not only by the increased strength, but also the reduced spring-back.

In one aspect of the invention, a surgical device includes a structural portion formed of a first element and having an outer surface disposed outwardly of an inner core. A second element is combined with the first element without creating a sure plane between the first element and the second element. This second element in combination with the first, provides the structural portion with a physical property which changes between the outer surface and the inner core. This physical property may be a co-efficient of friction, strength, or ductility, for example.

In another aspect of the invention, a method for manufacturing a surgical device includes the step of forming the surgical device of a metal having an outer surface and an inner core. The metal of the surgical device is exposed to an interstatial element which is driven into the outer surface of the metal to form a combination of the metal and the interstitial element which has a physical property that changes with progressive positions toward the core of the metal. The metal may include titanium while the interstitial element may include oxygen which is driven into the titanium under high temperature to create an oxygen gradient from the outer surface to the inner core.

In a further aspect, the invention includes a method for increasing the strength of a surgical device. The method includes the step of forming the device of a wire having an outer surface and an inner core. The wire is heated in the presence of an interstitial element which is driven into the outer surface of the wire. During the heating step, a concentration of the interstitial element is creased in the wire, the concentration being characterized by a first concentration at the outer surface and a second concentration less than the first concentration at the inner core.

### DESCRIPTION OF THE DRAWINGS

FIG. 1 is a schematic view of a leg of a patient illustrating the use of surgical clips for vessel occlusion;

FIG. 2 is a side elevation view of a surgical clip disposed in an open state relative to a vessel.

FIG. 3 is a side elevation view similar to FIG. 2 of the surgical clip disposed in a closed state relative to an occluded vessel;

FIG. 4 is a perspective view of an oven used in a process of the present invention;

FIG. 5 is a radial cross-section view of a clip showing a strength gradient associated with the present invention;

FIG. 6 is a schematic view of an apparatus used to test the pull force of a clip; and

FIG. 7 is a graph plotting pull force against temperature and time.

### DESCRIPTION OF PREFERRED EMBODIMENTS AND BEST MODE OF THE INVENTION

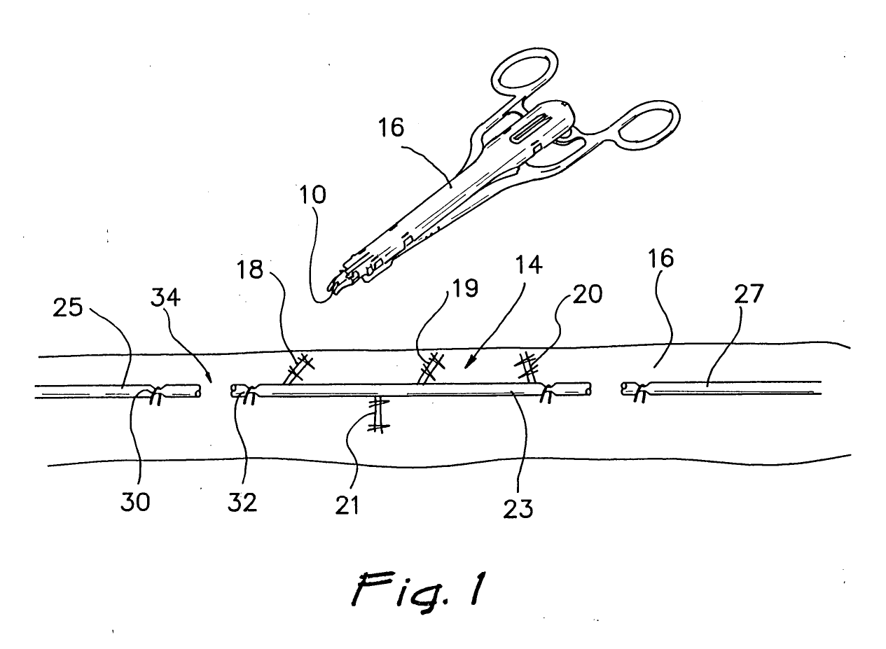

There are many surgical products made of metal which can benefit from the concept of the present invention. One such product is a surgical clip illustrated in FIG. 1 and designated by the reference numeral 10. The clip 10 is typically implemented with a clip applier 12 which initially holds the clip in an open state and which is operable to move the clip to a closed state. With these characteristics, the clip 10 and clip applier 12 are commonly used to occlude body conduits such as a blood vessel 14. In this process, the clip applier 12 is used to position the clip 10 in its open state over the vessel 14 and then to move the clip 10 to its closed state, thereby occluding the vessel 14. As long as the clip applier 12 is present to hold the clip in its closed state, the vessel 14 remains occluded. But eventually, the clip applier 12 must be removed. At this point in time, the continued occlusion of the vessel 14 is dependent solely on the strength of the clip 10.

In a surgical procedure referred to as coronary bypass, the vessel 14 may be the saphenous vein in a leg 16 of a patient. In a sub-procedure associated with the operation, the saphenous vein 14 is removed from the leg 16 to provide conduits for the coronary bypass. The saphenous vein 14 in the leg 16 is, of course, connected to many tributary veins 18-21, which must be occluded and severed in order to remove the saphenous vein 14. Furthermore, in order to remove a central portion 23 of the vein 14, that portion 23 must be severed from a distal portion 25 as well as a proximal portion 27 of the vein 14.

In a typical procedure, a pair of the clips 10 are applied to the vessel 14 and a cut is made between the two clips to sever the central portion 23 from the distal portion 25. For example, with reference to FIG. 1, a clip 30 and a clip 32 might initially be applied to the saphenous vein 14. A cut 34 can then be made between the clips 30 and 32 in order to sever the central portion 23 of the vein 14 from the distal portion 25. With this procedure, the clips 30 and 32 inhibit blood flow from the distal portion 25 of the vein 14, as well as the central portion 23 of the vein 14, respectively.

This two-clip procedure can be used to sever the central portion 23 from the proximal portion 27 of the vein 14. In addition, the two-clip procedure can also be used to sever each of the tributaries 18-21 from the central portion 23 of the vein 14. Having severed all connections to the central portion 23 of the vein 14, that section of the vein 14 can now be removed for further processing and use in the coronary bypass operation.

This is just one of many procedures where a clip, such as the clip 10, can be used to occlude a body conduit, such as a vessel 14. Once the clip is in place and moved to its closed state, its strength is relied on to maintain occlusion of the conduit. Critical problems with bleeding and other types of leakage result when clips do not have the internal strength necessary to maintain the occlusion.

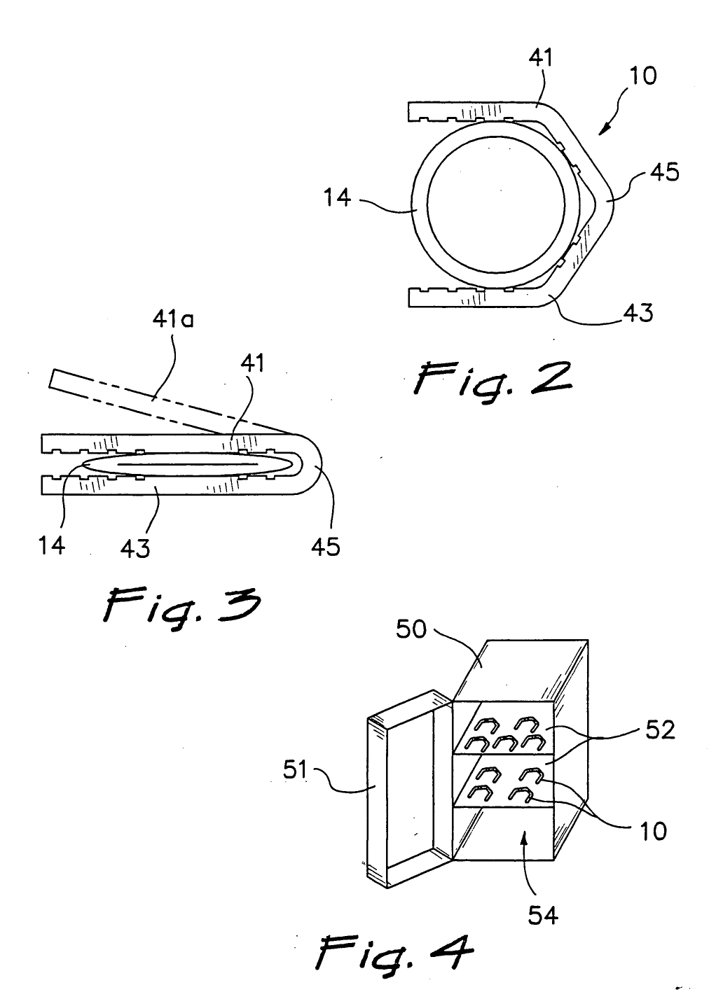

The process of clip implementation is illustrated in greater detail in FIGS. 2 and 3. In FIG. 2, the clip 10 is illustrated to be formed of a titanium wire and to be provided with a pair of elongate legs 41 and 43 which are connected at one end to an elbow 45. In the open state of the clip 10, the legs 41 and 43 are widely separated at their ends opposite to the elbow 45. This gives the clip 10 a “V” or “U” shape facilitating the disposition of the clip 10 over the vessel 14. When operatively positioned, the clip 10 can be moved to its closed state as illustrated in FIG. 3 wherein the legs 41 and 43 are brought into close proximity, pinching the vessel 14 closed. At this point, the clip applier 12 (FIG. 1), can be removed, leaving the clip 10 in the closed state and the vessel 14 in the occluded state.

One problem associated with this process relates to the stresses which are developed primarily at the elbow 45, as the legs 41 and 43 are bent from the open state to the closed state. These stresses tend to resist the bending and ultimately can result in spring-back, wherein the legs 41 and 43 open slightly as the clip applier 12 is removed. By way of illustration, the leg 41 may separate from the leg 43 slightly as shown by the dotted line 41a in FIG. 3. Of course it is desirable that spring-back be minimized in order that the close spacing of the legs 41 and 43 continued to occlude the vessel 14 even after the clip applier 12 is removed.

In accordance with the present invention, the clip 10 is exposed to a treatment process which greatly increases the strength of the clip and tends to minimize the spring-back of the clip 10. In a preferred process, an oven 50 is provided with a door 51 and a plurality of trays 52. The clips 10 are preferably placed on the trays 52 in a non-contacting relationship so that each clip is exposed to the air and particularly the oxygen within the oven 50. When the door 51 of the oven 50 is closed, the air within the oven 50 provides a source of oxygen 54, which functions as an interstatial element. This clearly distinguishes processes used in the past when every attempt has been made to evacuate the oven 50 in order to remove any oxygen from a heating environment.

With the door 51 closed, and the clips 10 exposed to the source of oxygen 54, the temperature of the oven 50 can be elevated. This will have the effect of driving or otherwise transferring the oxygen molecules from the source of oxygen 54 into the titanium of the clips 10. There are many changes in the environmental conditions which can have this effect. Increasing the concentration of oxygen in the source 54, or raising the temperature, the pressure or any other environmental conditions within the oven 50, may have the effect of facilitating the transfer of oxygen molecules into the titanium of the clips.

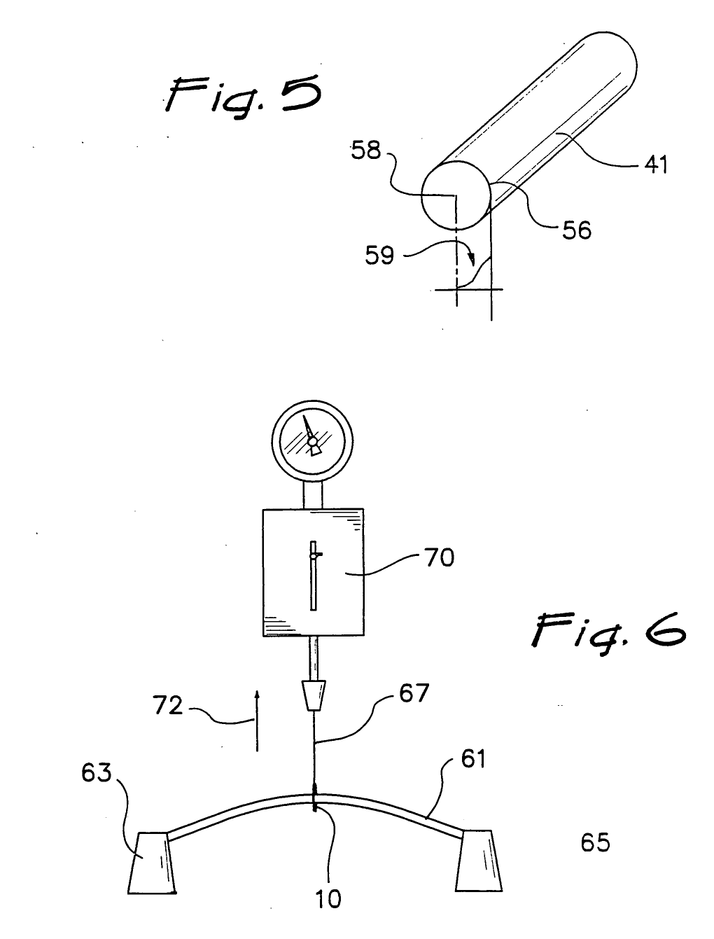

The advantage of this process, particularly with respect to a wire configuration, is illustrated in the radial cross-section view of FIG. 5. In this view, a cylindrical wire, such as that forming the leg 41 of the clip 10 is shown in a radial cross-section view. In this view, it can be seen that the leg 41 will typically have a cylindrical outer surface 56 as well as an inner core 58. As the clips 10 are processed in the oven 50 or other container, the oxygen from the oxygen source 54 is driven into the outer surface 56 and toward the inner core 58 of the wire or leg 41. With the greatest exposure to the oxygen source 54 at the outer surface 56, the highest concentration of oxygen molecules within the titanium tends to occur in proximity to this surface 56. It follows that the lowest concentration of oxygen molecules in the titanium occurs in proximity to the core 58.

In fact, it has been found that a gradient 59 of oxygen atom concentration within the titanium tends to exist between the outer surface 56 and the inner core 58. Where the oxygen concentration is the greatest, titanium oxide is formed which tends to have higher strength characteristics than does the pure titanium. Thus, in accordance with this process, the strength of the wire or leg 41 is increased primarily in proximity to the surface 56, while the more malleable characteristics associated with less oxidized titanium tends to remain in proximity to the inner core 58.

This strength gradient 59 can be adjusted by varying the extent to which the oxygen molecules are driven into the titanium, from the outer surface 56 toward the inner core 58. Variables which effect this strength gradient might include, for example, a greater concentration of oxygen atoms within the source 54, the amount of time the clips remain within the container or oven 50, and the driver (such as temperature or pressure) which is applied during the process.

In a specific case, the clip 10 is formed of a titanium (Grade 1) wire having a diameter of 0.027 inches or a rectangular cross section of 0.020×0.035 inches, for example The clip is then heated to an elevated temperature such as 975° F., in the presence of air which of course includes oxygen and nitrogen. These interstitial elements are driven into the interstitial spaces of the titanium forming the gradient 59 of primarily oxygen concentration which decreases inwardly from the surface 56 of the clip 10. This gradient 59 is thought to be of particular importance to the present invention.

It is generally known that the more interstitial oxygen present in titanium, the greater its strength and yield, but the less its ductility. This interstitial oxygen, as well as the addition of iron, are part of the criteria which control the hardness in different grades of titanium. Titanium (Grade 1), for example, is known to have the least amount of oxygen and therefore the lowest strength and highest ductility. Grades 2, 3 and 4 advance to the highest concentration of oxygen and the greatest strength. Unfortunately, the higher grades also exhibit decreased ductility and increased brittleness.

Thus, titanium in these four grades has been offered with a homogenous oxygen concentration in each grade. No gradient of oxygen concentration has been produced nor has there ever been any appreciation that a highly controllable gradient might provide an ultimate compromise between strength, ductility and brittleness.

The gradient 59 is of further interest because the oxygen concentration decreases gradually from the surface 56 to the core 58. There is no fracture plane that exists between the surface and the core. As a result, there is no cracking, flaking, or any embrittlement associated with the present invention.

The strength of the clip 10 can be carefully controlled to provide the most advantageous properties for the clip 10. Realizing that both strength and ductility are of interest to the clip 10, it is desirable that the added strength be carefully controlled.

In one specific process, titanium (Grade 1) is extruded to form a wire which is then bent to the configuration desired for the clip 10. The bent clip 10 is then heated in a vacuum in order to reduce the stresses left from the bending step. Then the clip is subjected to an elevated temperature such as about 975° F. for a period of about 15 minutes in order to obtain the desired gradient. In general, it has been found that an increase of about 10° F. in temperature will double the diffusion rate of the interstitial oxygen. Thus, temperature variations offer a high degree of control over the surface concentration and gradient.

In addition to temperature variations, the time during which the elevated temperature is applied can also be controlled. It has been found that when the time is increased by a factor of four, the diffusion rate of oxygen is approximately doubled.

It should be noted that in the teachings of the past, the intentional application of oxygen to titanium is to be strictly avoided. The resulting interaction between titanium and air at elevated temperatures has been thought to produce undesirable oxidation and corrosion. Present literature tends to deal with this problem by further teaching how to prevent oxygen penetration beyond the natural layer.

Notwithstanding these teachings of the past, the present invention benefits from the change in physical and chemical properties of heat treatment in an ambient air environment. Competing physical properties such as ductility, surface finish, and coefficient of friction (to name a few) can be optimized for the intended application.

Although the art has long appreciated the role of oxygen, nitrogen, and carbon in altering the properties of titanium, it has not appreciated the dramatic effect of subtle variations in these properties. In fact, clip manufacturers have gone to great lengths to limit themselves to titanium (Grade 1), in order to avoid potential problems caused by titanium (Grade 2). They have not considered the infinite number of variations that can be created within titanium (Grade 1), due to changes in chemical composition or even the distribution of chemicals within the metal.

In order to measure the strength of any clip, a pull test can be developed with apparatus such as that illustrated in FIG. 6. In this view, a silicone tube 61, representative of the vessel 14, can be stretched between a pair of clamps 63 and 65. The clip 10 is moved to its closed state as illustrated in FIG. 3 to occlude or clip the tube 61. A thin wire 67 can then be attached to the elbow 45 of the clip 10 and fixed to a force gauge 70. As the force gauge 70 is moved away from the tube 61, an increasing force is applied to the elbow 45 which tends to remove the clip 10 from the tube 61. This force, measured by the gauge 70, is applied in the direction of an arrow 72 illustrated in FIG. 6. The amount of force required to remove the clip 10 from the tube 61 becomes a measure of the strength of the clip 10 in its closed position.

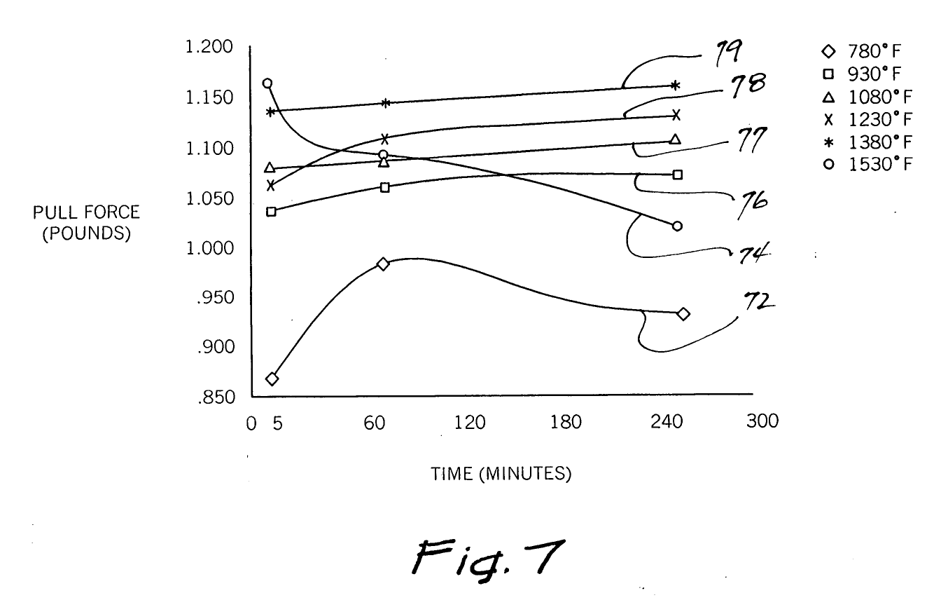

Using the pull test illustrated in FIG. 6, the strength of the clips 10 can be measured with variations in the parameter of the treatment process. A plot of the pull force against time at different temperatures is set force in FIG. 7. It will be noted that in this case, the clip 10 was formed with a wire diameter of 0.027 inches and the legs 41 and 43 at a length of about 0.275 inches. The silicone tubing 61 used in the pull test had an outside diameter of 0.094 inches, and an inside diameter of 0.031 inches.

The graph of FIG. 7 readily shows a preferred range of times and temperatures. At one extreme a plot, designated by the reference numeral 72, shows a change in pull force when the clips were processed at a temperature of 780 degrees. When the process duration was 15 minutes, this plot 72 shows a relatively low strength which increased as the process duration was extended to 60 minutes. But even this strength did not achieve levels associated with higher temperatures. At the opposite extreme, a plot 74 shows the strength of clips processed at a temperature of 1530° F. While this strength reached its highest level with a process duration of 15 minutes, the strength decreased rather dramatically with further increases in the duration of the process.

Between these two extremes, a range of temperatures and times developed the plots 76, 77, 78, and 79. Each of these plots is relatively linear and provides a relatively high strength characteristic. Among these four plots in the preferred range, the highest strength was demonstrated by the plot 79 which was associated with a process temperature of 1380° F. This strength degraded only slightly with temperatures down to 930° F. where, with a process duration of 15 minutes, the clips 10 had a pull force or strength of about one pound. These parameters are illustrated in the plot 76. In general, the process parameters in this preferred range include a temperature between about 930° F. and about 1380° F., and a process duration between about 15 minutes and about 240 minutes. By comparison, it will be noted that the same clips, without the processing of the present invention, might have a pull force of 0.635 pounds.

The resulting titanium oxide and/or increased oxygen concentration at the surface 56 of the clip 10 appears to create a reduced coefficient of friction. This will facilitate movement of the clips 10 through the feed channel of the clip applier 12 (FIG. 1). Notwithstanding this fact, the titanium oxide surface is probably rougher than that of pure titanium so that its retention characteristics with respect to tissue may actually increase. In this context, retention is probably dependent more on the shear forces associated with the titanium/tissue interface, than with the actual coefficient of friction. Thus, the titanium oxide surface may actually provide the clip 10 with two advantages associated directly with the surface characteristics of the clip.

From a manufacturing standpoint, process time becomes a consideration. Lengthy processes tend to add to the cost of a product, so the shorter processes are preferred. Thus, the strength characteristics at a process duration of 15 minutes might be of particular interest from a manufacturing standpoint.

Other considerations in choosing the most preferred process parameters might relate to the marketability of the product in terms of its appearance. In this regard, it was noted that the different temperatures and times tended to provide a variation in the color of the clips 10. As illustrated in the following table, a gold color occurred at lower temperatures and lower process durations.

TABLE I
Clip Color at Different Temperatures

| TEMPER- | | | |
|---|---|---|---|
| ATURE | 15 MINUTES | 60 MINUTES | 240 MINUTES |
| 780 | Gold | Gold | Gold |
| 930 | Gold | Gold/brown | Bright purple |
| 1080 | Purple/gold | Light blue | Light green/blue |
| 1230 | Silver/light blue | Purplish blue green | Dark gray |
| 1380 | Gray | Gay | Gray |
| 1530 | Gray | Light gray | White |

As can be seen from Table I, the gold color tended toward brown, blue and gray with increased temperatures and times. With a preference for the gold color, it was determined that the best parameters for a particular process might be achieved with an oven temperature of about 930° F. and a process duration of about 5 minutes. In accordance with the graph of FIG. 7, this would provide a pull strength of about 1 pound and provide the desired gold appearance.

Of course it will be appreciated that the oven 50 is representative of any processing chamber for the clips 10 which might include a source of oxygen 54 or other gas with a variable purity, and which could be used to apply a driver such as pressure or temperature in order to control the process. In this manner, the atoms provided by the source 54 can be driven into the metal of the object to provide a predetermined compromise between strength and ductility as required for a specific medical device.

It will be understood that many other modifications can be made to the various disclosed embodiments without departing from the spirit and scope of the concept. For example, various sizes of the surgical device are contemplated as well as various types of constructions and materials. It will also be apparent that many modifications can be made to the configuration of parts as well as their interaction. For these reasons, the above description should not be construed as limiting the invention, but should be interpreted as merely exemplary of preferred embodiments. Those skilled in the art will envision other modifications within the scope and spirit of the present invention as defined by the following claims.

1. A surgical device, comprising:
a structural portion formed of a first element and having an outer surface and an inner core;
a second element combined with a first element without creating a shear plane between the first element and the second element;
the second element, with the first element providing the structural portion with a physical property which changes between the outer surface and the inner core of the structural portion.

2. The surgical device recited in claim 1, wherein:
the physical property is a coefficient of friction; and
the coefficient of friction decreases between the outer surface and the inner core.

3. The surgical device recited in claim 2, wherein the coefficient of friction decreases with progressive positions towards the outer surface.

4. The surgical device recited in claim 1, wherein the second element is impregnated into the first element in an amount which decreases from the outer surface to the inner core.

5. The surgical device recited in claim 1, wherein the first element is a metal.

6. The surgical device recited in claim 2, wherein the second element is a gas.

7. The surgical device recited in claim 1, wherein the surgical device comprises a clip.

8. The surgical device recited in claim 6, wherein the metal is titanium and the gas is oxygen.

9. The surgical device recited in claim 1, wherein the physical property is strength, and the strength changes between the outer surface and the inner core.

10. The surgical device recited in claim 9, wherein the strength changes in a gradient which decreases with progressive positions toward the inner core.

11. A method for manufacturing a surgical device, including the steps of:
forming the surgical device of a metal having an outer surface and an inner core;
exposing the metal of the surgical device to an interstitial element; and
driving the interstitial element into the outer surface of the metal to form a combination of the metal and the interstitial element which has a physical property that changes with progressive positions toward the core of the metal.

12. The method recited in claim 11, wherein during the driving step, the method further comprises the steps of:
creating a gradient of the interstitial element in the metal, the gradient having a concentration of the interstitial element in the metal which decreases from the other surface toward the inner core.

13. The method recited in claim 11, wherein the metal includes titanium.

14. The method recited in claim 13, wherein the interstitial element includes oxygen.

15. The method recited in claim 12, wherein the driving step includes the step of heating the metal and the interstitial element.

16. The method recited in claim 12, wherein the driving step includes the step of pressurizing the metal and the interstitial element.

17. The method recited in claim 11, wherein the physical property includes at least one of friction, strength and ductility.

18. The method recited in claim 17, wherein:
the strength increases with progressive positions toward the outer surface; and
the ductility increases with progressive positions toward the inner core.

19. A method recited in claim 12 wherein the surgical device is one of a clip, clamp, needle and staple.

20. A method for increasing the strength of a surgical device, comprising the steps of:
forming the device of a wire having an outer surface and an inner core;
heating the wire in the presence of an interstitial element to drive the interstitial element into the outer surface of the wire; and
during the heating step, creating in the wire a concentration of the interstitial element, the concentration being characterized by a first concentration of the interstitial element at the outer surface and a second concentration of the interstitial element at the inner core; and
the first concentration of the interstitial element being greater than the second concentration of the interstitial element.

21. The method recited in claim 20, wherein the creating step includes the step of:
creating a concentration gradient in the wire which varies progressively between the first concentration at the outer surface and the second concentration at the inner core.

22. The method recited in claim 20, wherein prior to the heating step, the method includes the step of coating the wire with a material including the interstitial element.

23. The method recited in claim 22, wherein the heating step includes the steps of:
heating the coating material to a temperature and for a time sufficient to generally free the interstitial element from the coating material; and
heating the wire to a temperature and for a time sufficient to drive the freed interstitial element into the wire.
